(19) United States
(12) Patent Application Publication
(10) Pub. No.: US 2022/0143340 A1
(43) Pub. Date: May 12, 2022

(54) NASAL TRACHEAL DEVICES AND RELATED METHODS

(71) Applicants: Uday Reebye, Chapel Hill, NC (US); Alexander Guizzotti, Chapel Hill, NC (US); Andrew Lees, Wake Forest, NC (US)

(72) Inventors: Uday Reebye, Chapel Hill, NC (US); Alexander Guizzotti, Chapel Hill, NC (US); Andrew Lees, Wake Forest, NC (US)

(21) Appl. No.: 17/521,304

(22) Filed: Nov. 8, 2021

Related U.S. Application Data
(60) Provisional application No. 63/247,329, filed on Sep. 23, 2021.
(60) Provisional application No. 63/113,135, filed on Nov. 12, 2020.

(51) Int. Cl. A61M 16/04; A61M 16/08

(52) U.S. Cl. CPC A61M 16/0461; A61M 16/0488; A61M 2205/584; A61M 16/0891; A61M 16/0434

(57) ABSTRACT

Nasal tracheal devices include a tube with visual indicia of insertion depth corresponding to patient height and an inflatable cuff. The tube comprises or provides a plurality of lumens that are in fluid isolation including an airpath lumen and a pressurized fluid source for inflating the cuff. The devices may also be configured to suction patient fluid from a location adjacent and/or above the cuff.

### RELATED APPLICATION(S)

This patent application claims the benefit of and priority to U.S. Provisional Application Ser. No. 63/247,329, filed Sep. 23, 2021, and U.S. Provisional Application Ser. N. 63/113,135, filed Nov. 12, 2020, the contents of which are hereby incorporated by reference as if recited in full herein.

### FIELD OF THE INVENTION

The present invention relates to medical intubation devices.

### BACKGROUND

Surgical procedures or medical treatments sometimes require intubation. Intubation is the placement of a tube in a patient's throat to help move air (oxygen blend) in and out of the lungs. Typically, intubation procedures require that the patient be placed under general anesthesia because insertion of the intubation tube may trigger the patient's pharyngeal reflex which may prevent proper placement of the tube. Intubation is usually performed endotracheally or through the use of a laryngeal mask airway.

Endotracheal intubation involves inserting a flexible plastic tube through the patient's mouth and into the trachea. At its proximal end, the tube is connected to a ventilator, which delivers oxygen to the lungs, allowing the patient to breathe. Once the tube is inserted and placed in the proper position, the tube takes up a significant portion of the oral cavity and creates an obstruction that the surgeon must try to work around.

Typically, the endotracheal tube is inserted with the assistance of a lighted laryngoscope. The scope is carefully threaded between the vocal cords and into the lower trachea. The surgeon must confirm that the tube is in proper position and not inadvertently placed in the esophagus, which is confirmed by observing chest movement or fogging in the tube. When the endotracheal tube is in proper position, a balloon cuff is inflated to keep the tube from moving out of place. The tube is then taped to the patient's face. The tube is then attached to a ventilator to provide oxygen to the patient and support breathing during sedation.

The risks associated with endotracheal intubation include injury to the mouth, teeth, tongue, thyroid gland, voice box, vocal cords, trachea or esophagus upon insertion or removal of the tube. Other risks include bleeding, infection, hoarseness after the tube is removed, pneumothorax (collapsed lung, which occurs when the tube is advanced too far, such that it only enter one lung), aspiration pneumonia (aspiration of the contents of the stomach) or lack of oxygen due to improper placement.

Another method, employing a laryngeal mask airway, also involves inserting a tube into the patient's mouth, however, here, the tube is positioned above the vocal cords. While this method eliminates the risk of damage to the vocal cords, it requires the use of a mask that covers the entirety of the patient's mouth and nose, thereby making it difficult or impossible to perform surgical procedures in the patient's oral cavity.

There is a need for alternative medical devices to improve safety and/or facilitate intubations.

### SUMMARY

Embodiments of the present invention are directed to nasal tracheal tubes that are color-coded to patient-height to allow for rapid and proper placement of a distal end portion of the tube above the vocal cords (supraglottic).

Embodiments of the present invention provide nasal tracheal tubes that can suction fluid adjacent a cuff of the nasal tracheal tube to prevent or reduce liquid that may otherwise be allowed to enter the lungs during intubation.

Embodiments of the present invention provide a cuff with a pear-shaped configuration to thereby reduce drag at withdrawal.

Embodiments of the present invention are directed to nasal tracheal devices. The devices include a tube with a distal end portion configured to reside inside a trachea of a patient. The tube has axially spaced apart proximal and distal end portions. The tube includes a segment that resides between the proximal and distal end portions that has visual indicia of insertion depth correlated to patient height. The device also includes an inflatable cuff integral to or coupled to the distal end portion of the tube. The inflatable cuff is sized and configured to be positioned supraglottic.

The tube can have a first lumen extending axially along the tube and a second lumen in fluid isolation with the first lumen and also extending axially along the tube. The first lumen can be in fluid communication with the inflatable cuff.

The visual indicia of insertion depth can include a plurality of bands of different colors.

The plurality of bands can include at least three bands of different colors, optionally 3-10 bands of different colors.

The inflatable cuff can have a pear-shaped configuration or include a pear-shaped segment when in an inflated state.

The inflatable cuff can be configured to define a fluid collection portion.

The tube can include a third lumen extending axially along the tube and the third lumen may be in fluid isolation from the first and second lumens.

The proximal end portion of the device can have at least one connector providing a first connection to the first lumen and a second connection to the second lumen.

The second lumen can be configured to connect to a ventilator, the first lumen can be configured to connect to an inflation fluid source and the third lumen can be configured to connect to a vacuum source whereby the first lumen defines an inflation path, the second lumen defines an oxygen path and the third lumen defines an aspiration path.

The tube can have a bend segment between the proximal and distal end portions. The bend segment can reside above the visual indicia of insertion depth and can have (extend at) an angle of between 90-120 degrees.

The third lumen can b provided at least in part by a conduit that extends external to the tube with a free end that resides adjacent the cuff

The conduit can be configured to reside against a wall of the inflatable cuff when the cuff is in a non-inflated state. The conduit can be configured to be pushed outward in response to inflation of the cuff.

The cuff can have a neck at an upper end thereof that merge into a bottom portion that can define a maximal lateral extent of the inflatable cuff when inflated. The maximal lateral extent can be 2-3 times greater than a (maximal) lateral extent of the neck when the inflatable cuff is inflated.

The inflatable cuff can have a radius of curvature that extends between the neck and the bottom portion that is in a range of 1-1.5 inches over 0.5-1 inch.

Other embodiments are directed to intubation systems that include a nasal tracheal device with a tube with an inflatable cuff. The tube has a distal end portion configured to reside inside a trachea of a patient above vocal cords of the patient. The tube can have axially spaced apart proximal and distal end portions. The tube can have a segment that resides between the proximal and distal end portions that includes visual indicia of insertion depth correlated to patient height. The tube has a first lumen extending axially along the tube and a second lumen in fluid isolation with the first lumen and also extending axially along the tube. The first lumen is in fluid communication with the inflatable cuff. The system also includes: a ventilator or anesthesia circuit in fluid communication with the second lumen of the tube; and an inflation fluid source in fluid communication with the first lumen of the tube to inflate the inflatable cuff.

The system can also include a third lumen provided by the tube and/or a conduit coupled to the tube and a vacuum in fluid communication with the third lumen and defining an aspiration path.

The inflatable cuff can have a pear-shaped body when in an inflated state.

The visual indicia of insertion depth can include a plurality of bands of different colors.

Yet other embodiments are directed to methods of placing a nasal tracheal tube in a patient. The methods include: determining or obtaining a height estimate of a patient; providing a nasal trachea device with a tube comprising an inflatable cuff and visual indicia of insertion depth correlated to different heights of patients; inserting the tube via an intranasal insertion path to an insertion depth in a trachea of the patient corresponding to the visual indicia associated with the estimated height of the patient thereby placing a distal end portion of the tube above the vocal cords (supraglottic) of the patient; and inflating the inflatable cuff so that the cuff seals against an inner wall of the trachea above the vocal cords.

The inserting can be carried out to place a color band associated with the estimated patient height at least partially external to a nostril of the patient.

The inflatable cuff can have a pear-shaped body or pear-shaped body segment with a larger portion defining a maximal lateral extent of the inflatable cuff when inflated below a smaller upper portion, wherein the larger portion is 2-3 times larger than the smaller upper portion when inflated.

The methods can also include suctioning liquid from the patient using a conduit provided by or coupled to the tube adjacent the inflatable cuff.

Further features, advantages and details of the present invention will be appreciated by those of ordinary skill in the art from a reading of the figures and the detailed description of the preferred embodiments that follow, such description being merely illustrative of the present invention.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE DRAWINGS

FIG. 1A is a side perspective view of an example nasal tracheal device according to embodiments of the present invention.

FIG. 1B is a schematic illustration showing an example placement of the cuff of the nasal tracheal device shown in FIG. 1A according to embodiments of the present invention.

FIG. 2 is a schematic illustration of a distal end portion of a nasal tracheal device according to some embodiments of the present invention.

FIG. 3A is a side perspective view of another example of a nasal tracheal device according to embodiments of the present invention.

FIG. 3B is a side perspective view of the device shown in FIG. 3A illustrating an insertion path according to embodiments of the present invention.

FIG. 4A is a greatly enlarged end perspective view of the distal end portion of the nasal tracheal device shown in FIG. 3A.

FIG. 4B is an enlarged side perspective view of another embodiment of the distal end portion of the nasal tracheal device according to embodiments of the present invention.

FIG. 5 is a greatly enlarged end perspective view of the nasal tracheal device shown in FIG. 3A.

FIG. 6 is a side perspective view of the nasal tracheal device shown in FIG. 3A.

FIG. 7A is another side perspective view of the nasal tracheal device shown in FIG. 6.

FIG. 7B is a schematic illustration of the nasal tracheal device shown in FIG. 7A but illustrating the cuff in deflated configuration according to embodiments of the present invention.

FIG. 8 is an enlarged section view taken along line 8-8 in FIG. 7A.

FIG. 9 is a side perspective view of an inflatable cuff (shown in an inflated configuration) for a nasal tracheal device according to embodiments of the present invention.

FIG. 10 is a distal end view of the cuff shown in FIG. 9.

FIG. 11 is a side view of the cuff shown in FIG. 9.

FIG. 12 is a section view of the cuff taken along line 12-12 in FIG. 11.

FIG. 13 is a flow chart of example actions for an intubation procedure according to embodiments of the present invention.

FIG. 14A is a table of an example patient height to color band correlations according to embodiments of the present invention.

FIG. 14B is a graph of the data (height/inches color band correlation) in FIG. 14B.

FIG. 15 is a table of color-coded parameters with patient height distributions and color band lengths and positions correlated to heights according to embodiments of the present invention.

FIG. 16A is a of patient height (inches) to color bands according to embodiments of the present invention.

FIG. 16B is a graph of the color bands/markers shown in FIG. 16A with length relative to length from a distal end of the intrabody tube for color-coded installation according to embodiments of the present invention.

FIG. 17 is a table of example color-band parameters for an intranasal-tracheal tube according to embodiments of the present invention.

Further features, advantages and details of the present invention will be appreciated by those of ordinary skill in the art from a reading of the figures and the detailed description of the preferred embodiments that follow, such description being merely illustrative of the present invention.

### DETAILED DESCRIPTION

The present invention now will be described more fully hereinafter with reference to the accompanying drawings, in which illustrative embodiments of the invention are shown. Like numbers refer to like elements and different embodiments of like elements can be designated using a different number of superscript indicator apostrophes (e.g., 10, 10′, 10″, 10″′).

In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath”, “below”, “lower”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90° or at other orientations) and the spatially relative descriptors used herein interpreted accordingly. The term “about” refers to numbers in a range of +/−20% of the noted value. Any numerical range stated to be between two numbers is inclusive of the end point numbers.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein.

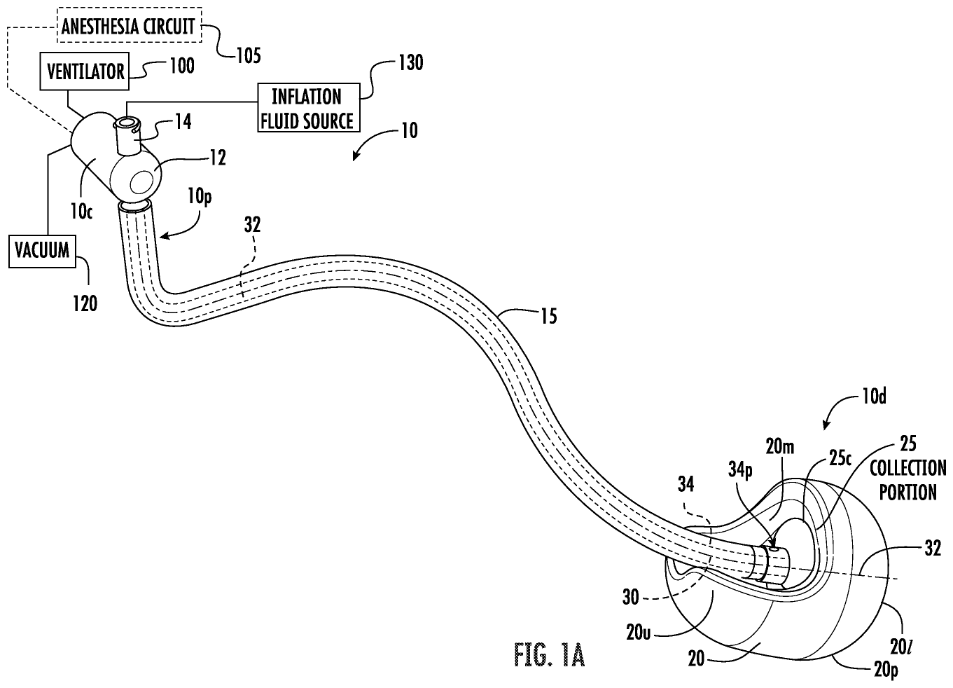

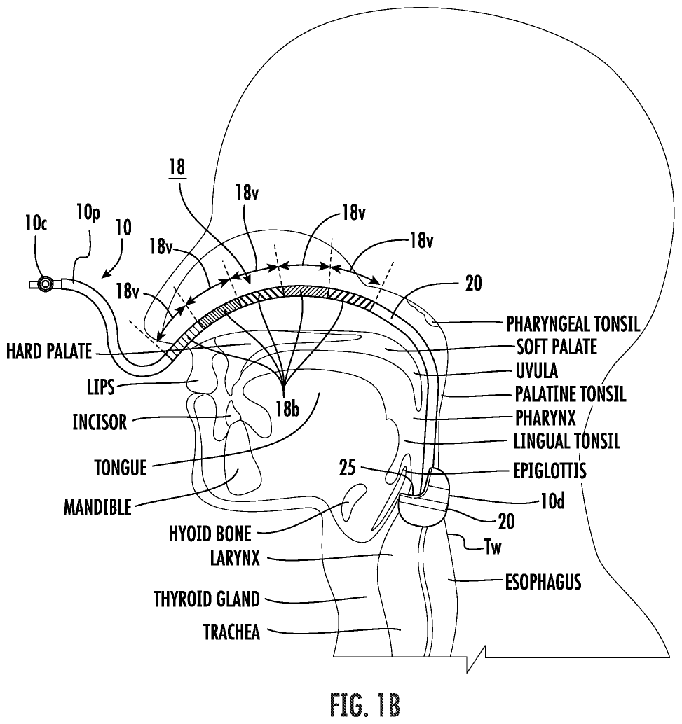

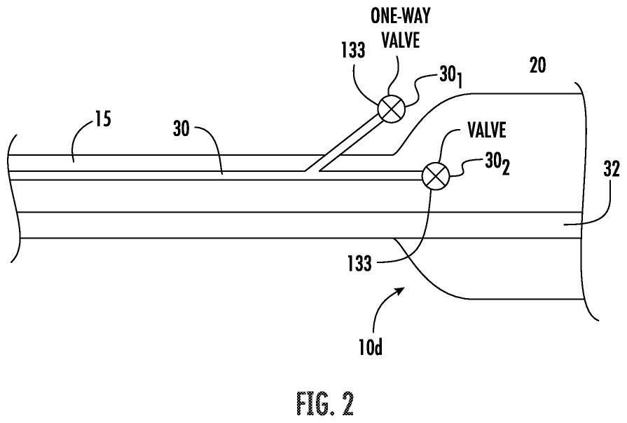

Turning now to FIG. 1A, an example nasal tracheal device 10 is shown. The nasal tracheal device 10 comprises opposing proximal and distal end portions 10p, 10d, respectively. The nasal tracheal device 10 comprises a tube 15 extending between the proximal and distal end portions 10p, 10d, respectively. The nasal tracheal device 10 includes a cuff 20 on the distal end portion 10d. The tube 15 comprises a first lumen 30 that is in fluid communication with the cuff 20 and an inflation fluid source 130. The pressurized fluid source 130 can be provided as a syringe with air or other medical grade gas and can be configured to inflate the cuff 20. The tube 15 comprises a second lumen 32 that provides an open delivery path to deliver oxygen from a ventilator 100 to the trachea, then to the lungs of the patient. The first lumen 30 is fluidly isolated from the second lumen 32. The second lumen 32 can alternatively be connected to an anesthesia circuit (105) for providing anesthesia.

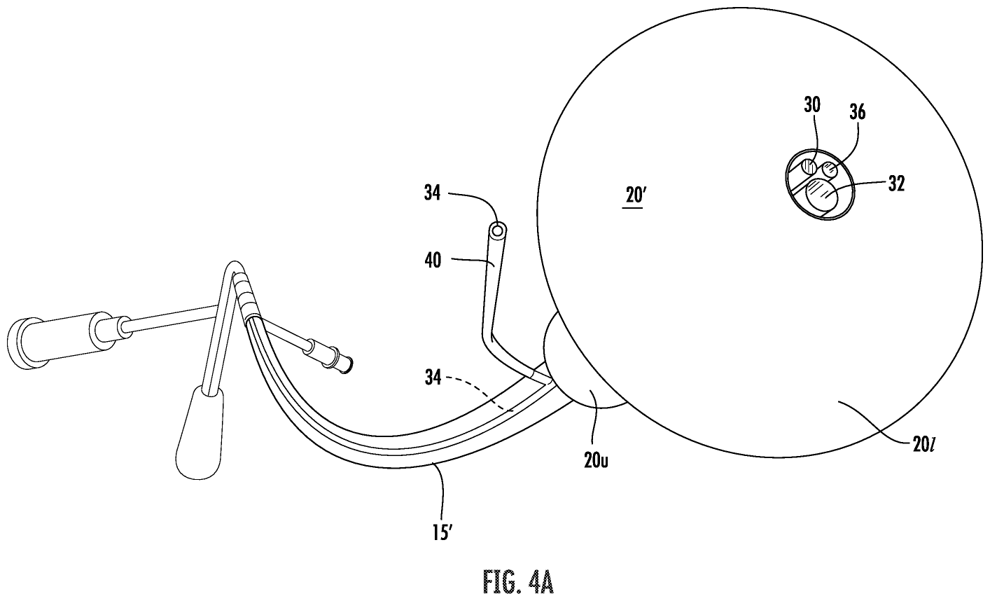

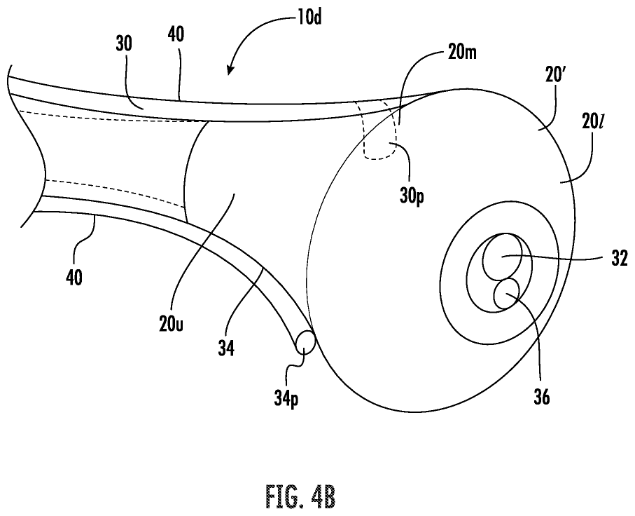

The tube 15 can comprise a third lumen 34 that extends between a suction source, such as a vacuum 120, to at least one suction intake port 34p. The at least one suction intake port 34p can reside adjacent the cuff 20, typically above the lower end portion 20l of the cuff 20, such as at or adjacent an upper end portion 20u or a medial 20m location of the cuff 20. The third lumen 34 can be fluidly isolated from the first and second lumens 30, 32. The first, second and third lumens 30, 32, 34 are shown in broken line in FIG. 1A as internal to the tube 15. However, one or both of the first lumen 30 and the third lumens 34 can be provided in one or more external conduits 40 coupled to the tube 15 (FIG. 4B).

In some embodiments, the first lumen 30 can be used to both inflate the cuff and to aspirate fluids during the procedure. For example, as shown in FIG. 1B, the first lumen 30 can be selectively coupled to a first port for the inflation and a second port for the aspiration. The first lumen 30 can be configured to branch at the cuff to provide the first and second ports 30₁, 30₂ and one-way valves 133 can be used to allow the aspiration or inflation.

Referring again to FIG. 1A, the proximal end portion 10p of the nasal tracheal device 10 comprises at least one connector 10c that is configured to engage the ventilator 100, optionally also the inflation fluid source 130 and/or the suction source/vacuum 120. As shown, the connector 10c can be provided as a single connector hub configured to connect to the ventilator 100, the vacuum 120 and the inflation source 130 and respective lumens.

The tube 15 is sized and configured to be insertable through the patient's nasal cavity, instead of through the oral cavity. Insertion through the nasal cavity allows intubation without obstructing the patient's oral cavity with a large foreign object, thereby allowing the surgeon to perform the required procedure without impediment. The patient is not required to be sedated or placed under general anesthesia for insertion of the tube through the nasal cavity. The nasal tracheal device 10 can be configured for human or veterinarian uses. The nasal tracheal device 10 can be used for ENT procedures, field, ambulance or EMT uses, hospital or field uses in military and civilian emergency medicine procedures, oral surgery, plastic surgery, and veterinarian procedures.

The nasal tracheal device 10 is configured so that the cuff 20 and the distal end 10d can be placed supraglottic, e.g., above the vocal cords. By positioning the device 10 above the vocal cords, risk of trauma to the vocal cords is eliminated or greatly reduced. Other potential side effects of endotracheal intubation are also eliminated or greatly reduced, including the risk of malignant hypothermia, since the patient is not required to be placed under general anesthesia.

Once in position, the cuff 20 can be inflated which inhibits and/or prevents fluids from entering the patient's lungs. The second lumen 32 of the tube 15 is attached to the ventilator 100 that continuously supplies oxygen to the lungs.

The cuff 20 can be sized and configured to seal the airway of the patient so that fluids do not enter the lungs outside the cuff 20. The cuff 20 can remain fully or partially deflated until it is in proper position, then the cuff 20 can be inflated using the inflation lumen 30. When inflated, the cuff 20 can abut and define a seal with a wall of the trachea Tw thereabout (FIG. 1B). It can be important to prevent fluid from passing below the cuff 20 (other than via lumens provided by the tube 15) because fluid that passes the cuff 20 may enter the lungs and cause infection and/or damage.

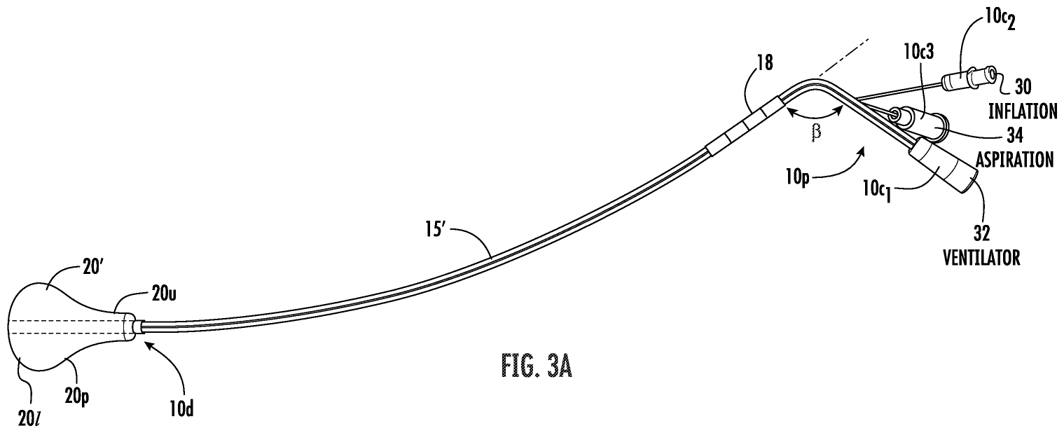

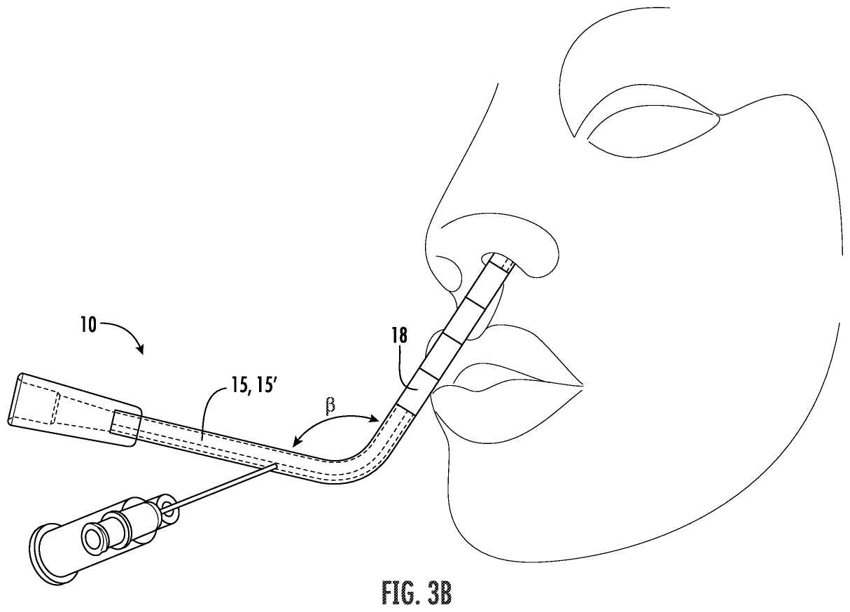

As shown in FIG. 1A, the cuff 20 can have a curvilinear profile 20p that tapers outward from a first lateral extent at the upper portion 20u to a wider lateral extent at the lower portion 20l. The cuff 20 can have a pear-shaped outer perimeter segment that may extend less than a full circumference of the body of the cuff. FIG. 3A illustrates that the cuff 20′ does not require a collection portion and the cuff 20′ can have a pear-shaped body. Unlike conventional cylindrical or elliptical cuffs, the cuff 20, 20′ can allow for reduced drag at withdrawal and/or induce less inflammation after deflation at withdrawal and/or can provide positive pressure to prevent travel over the vocal cords.

Referring to FIG. 1A, the cuff 20 can contain a collection portion 25 for collecting fluids. For example, fluid such as saliva from the patient's mouth and/or nasal discharge can be captured in the collection portion 25 of the cuff 20, and which can be aspirated (suctioned) periodically or continuously through suction into the intake port 34p of the third lumen 34. The collection portion 25 can comprise a channel 25c with a recess that faces the upper portion 20u of the cuff 20 and the channel 25c can extend circumferentially about at least a portion of the tube 15.

The third lumen 34 (e.g., “aspiration” or “suction” lumen) can be provided by the tube 15 as an internal lumen 34 or as an external conduit 40 with the lumen 34 or combinations of internal and external lumen segments that can extend over at least a major portion of a length of the tube 15.

Referring to FIG. 1B, in some embodiments, the tube 15 comprises a segment 18 with visual indicia 18v of a depth position of the distal end 10d of the nasal tracheal device 10 for placement based on height of a patient. The segment 18 extends over a sub-length of the tube 15 and can reside a distance from the proximal end portion 10p of the nasal tracheal device 10 and a distance from the distal end portion 10d of the nasal tracheal device 10. In use, the visual indica 18v corresponding to the height of the patient will reside adjacent, and at least partially external to, the intake zone of the nasal cavity thereby allowing a user to easily identify when the distal end portion 10d of the nasal tracheal device 10 is at a desired intrabody location.

The visual indicia 18v can be provided to identify a plurality of different height ranges, typically in a range of three-six, shown as four different height ranges. The visual indicia closest to the proximal end portion 10p of the nasal tracheal tube 10 can correlate to a tallest patient height range and the visual indicia furthest away from the proximal end portion 10p can correlate to a shortest height range.

The visual indicia 18v can comprise a plurality of bands 18b of different color. The bands 18b can be extend circumferentially about an entire external circumference of the tube or only about a portion of the tube 15. The bands 18 can be provided in solid or patterned colors. The bands 18b of color are color coded to visually indicate approximately how far the tube 15 should be inserted, depending on the height of the patient. As shown, the bands 18b of different color are provided in red, blue, green and yellow. Other colors may be used and/or other color orders may be used. In some embodiments, other or additional visual indicia of suitable depth can be used, such as numerical or textual markings, or alphanumeric markings or graduated scaled markings and the like can be provided to indicate approximately how far the tube 15 should be inserted.

The nasal tracheal device 10 can be provided in pediatric and adult sizes with the visual indicia 18v modified to correlate height to position based on the target end use.

Thus, the tube 15 can be inserted via an intranasal insertion path to an insertion depth in a trachea of the patient corresponding to the visual indicia associated with the estimated height of the patient thereby placing a distal end portion of the nasal tracheal device 10 and the tube 15 above the vocal cords (supraglottic) of the patient and then the inflatable cuff 20 can be inflated so that the cuff 20 seals against an inner wall of the trachea above the vocal cords. As shown in FIG. 1B, the tube 15 can be inserted to place a color band associated with the estimated patient height at least partially external to a nostril of the patient.

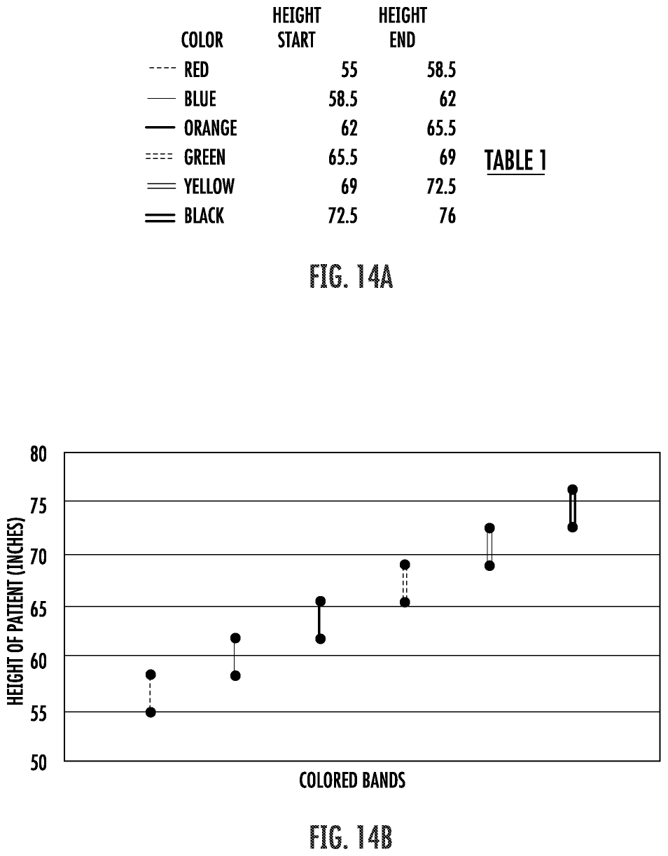

FIGS. 14A, 14B, 15, 16A, 16B and 17 provide example band placement based on height. The number of example bands identified is six but the number of bands can be lesser or greater, typically in a range of 3-10. FIG. 14A is a table, Table 1, of six color bands and FIG. 14B is a graph of the color bands correlated to patient height, with black used for a person in the highest height range and red used for a person in the lowest height range. The colors may be correlated for different segments or used in a different order, e.g., red can be used for the tallest and black for the shortest. The height data used to generate the correlation was obtained from the website in September, 2021: https://dqydj.com/height-percentile-calculator-for-men-and-women/ which collected data from the Centers for Disease Control (“CDC”) and only includes American heights. It is contemplated that different countries, genders, cultures, age groups and the like may have different height ranges and the positioning of the bands or other color indicia can vary for different patient populations.

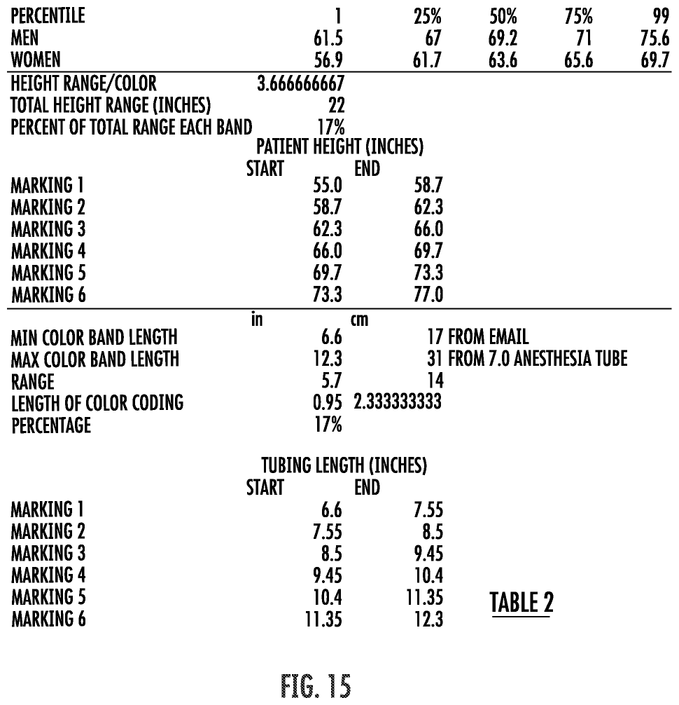

FIG. 15 is a table, Table 2, of color indicia data including percentile height distribution for male and female (inches) with a height range per color band of about 3.7 inches and a span of about 22 inches of height variation with markings 1-6 having start and end lengths (inches) of patient height. FIG. 15 also provides example minimum and maximum color band length, length of color coding and marking 1-6 start and end position relative to tubing length.

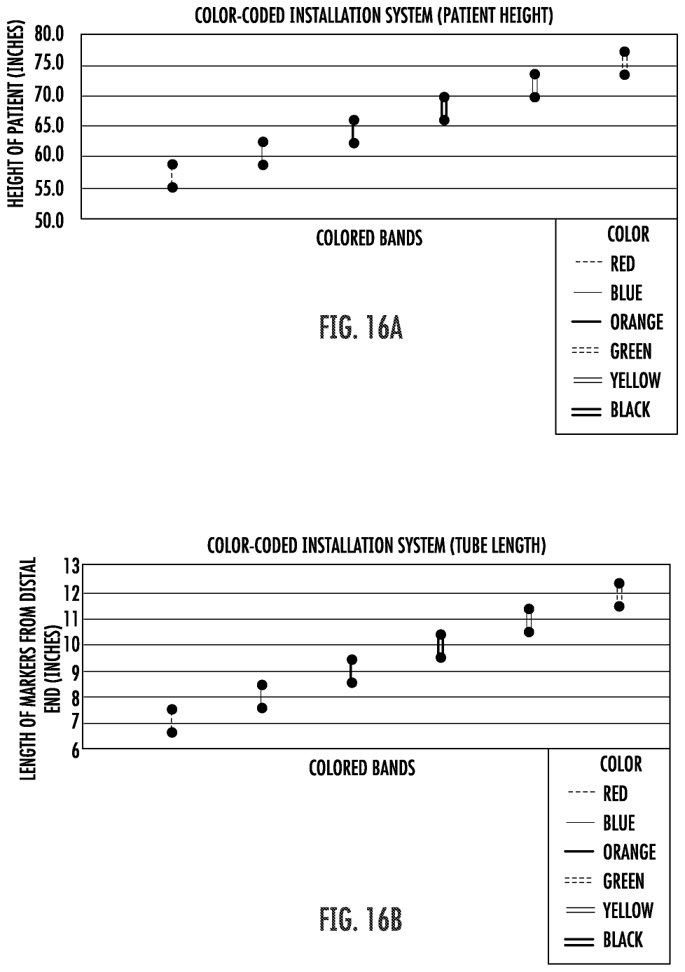

FIGS. 16A, 16B are example graphs with a different order of color bands (green for tallest and red for shortest) for a color-coded installation system. FIG. 16A correlates the patient height to color band. FIG. 16B shows a length of the color markers from a distal end of the tube (inches) according to some example embodiments.

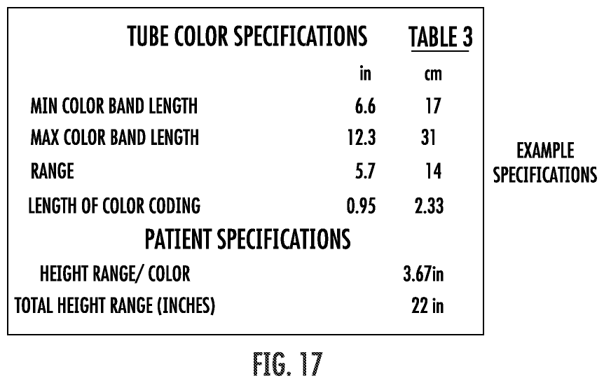

FIG. 17 is a table, Table 3, of example tube color parameters including min and max color band lengths, range and length of color coding, a height range per color band of about 3.67 inches and a total height range covered by the color coding of about 22 inches.

Referring again to FIG. 1A, the tube 15 can have a curvilinear elongate shape prior to implantation and can allow for a change in curvature during implantation. The curvature of the tube 15 can allow the tube to gently be inserted into the patient, so that it is more convenient to connect the air and suction hookup away from the patient's mouth and out of the way of the surgeon.

Referring to FIG. 3A, the at least one connector 10c at the proximal end portion of the nasal tracheal tube 10 can be provided as three separate connectors 10c₁, 10c₂, 10c₃. The connector 10c₁ for the ventilator 100 can be configured as an extension or end segment of the tube 15. The other connectors 10c₂, 10c₃ can be provided via conduits that branch off the tube 15 or that otherwise are coupled to lumens 30, 34, respectively.

FIGS. 3A and 3B also illustrate that the tube 15′ can be configured with a bent segment 15b above the visual indica segment 18 and before the proximal end with the connectors 10c₁, 10c₂, 10c₃. The bent segment 15b can have an angle β in a range of 100-120 degrees, typically about 110 degrees. The bent segment 15b can be bent to angle outward from the visual indicia segment 18 at a location that is at the maximum insertion depth provided by the device 10. The bent segment 15b places the connector 10c₁ at a position that allows a user to visually access a sight bore lumen 36 (FIGS. 4A, 4B).

Referring to FIG. 4A, in some embodiments, the suction lumen 34 can extend out of the tube 15 to an external conduit 40 at a location adjacent the upper portion 20u of the cuff 20′. This conduit 40 can be configured to reside against an outer surface of the cuff 20′ in a non-inflated state of the cuff to facilitate insertion of the nasal tracheal tube 10 into the nasal cavity and trachea. The inflation of the cuff 20′ can push the conduit 40 outward.

Referring to FIG. 4B, as discussed above, the nasal tracheal tube 10 can include one or more external conduits 40 that can merge into internal lumens or remain as external conduits providing the lumen 30 or 34. The intake port 34p can reside external to the tube 15. The inflation port 30p can be in fluid communication with the cuff 20′. FIG. 4B also shows the primary lumen 32 can reside adjacent a sight bore lumen 36.

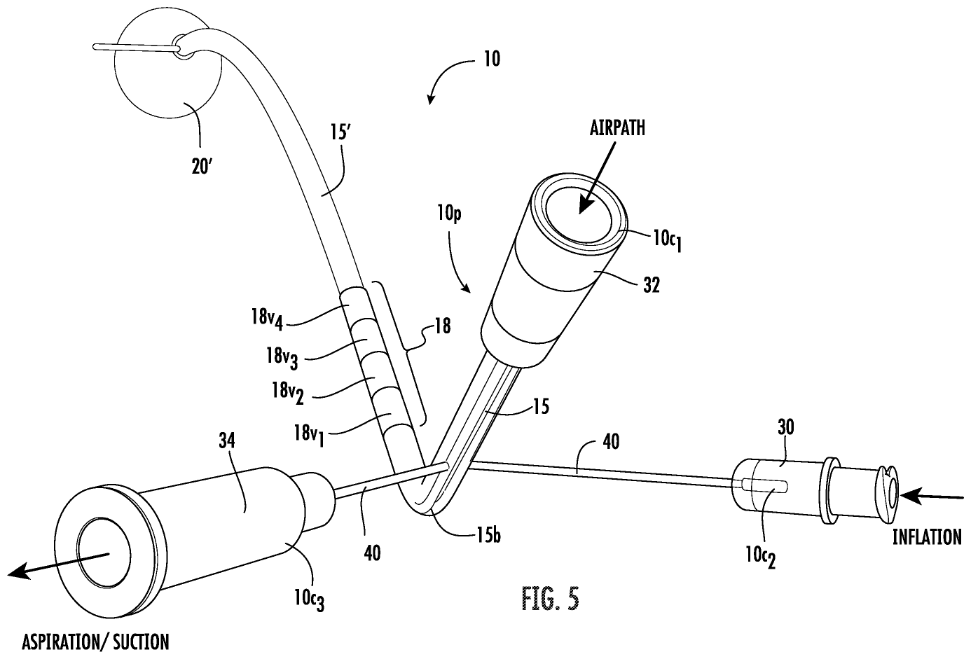

FIG. 5 illustrates the proximal end portion 10p of the nasal tracheal tube 10 and four different visual indicia segments 18v₁, 18v₂, 18v₃, 18v₄ providing the visual indicia segment 18.

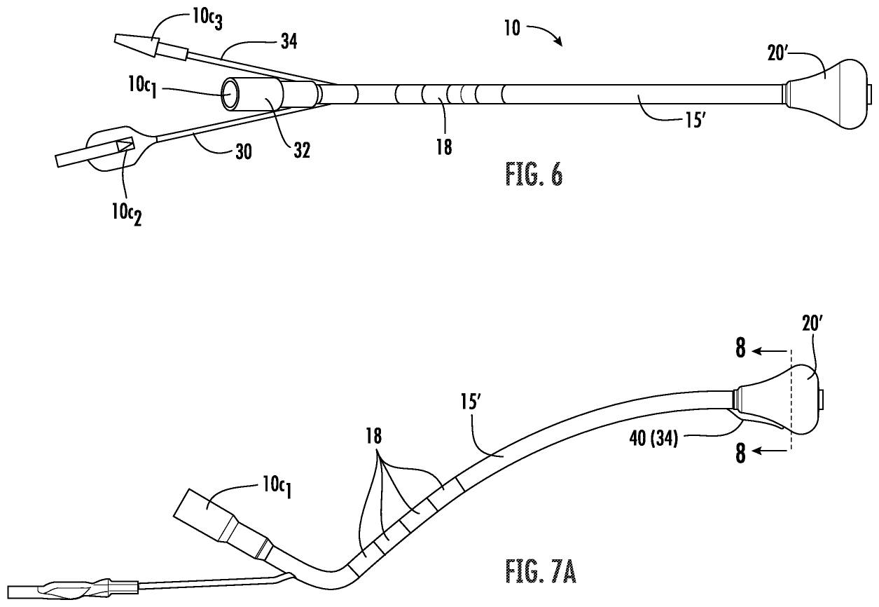

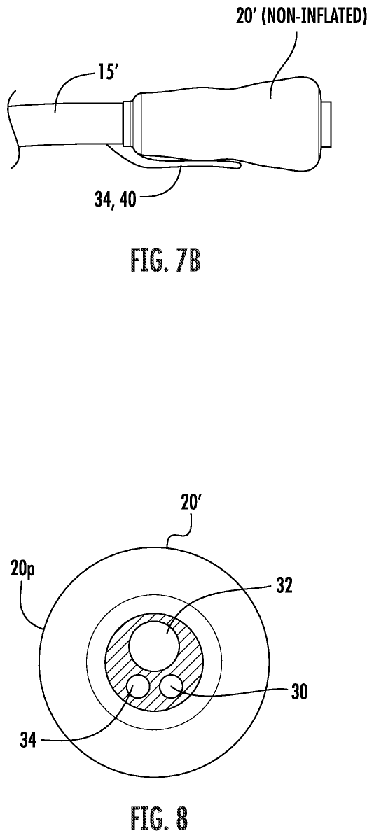

FIGS. 6 and 7A illustrate the nasal tracheal tube 10. FIG. 7B illustrates the conduit 40 providing the suction lumen 34 parallel with the tube 15 when the cuff 20′ is deflated (prior to inflation). FIG. 7A illustrates that the conduit 40 can be pushed radially outward by inflation of the cuff 20′.

FIG. 8 illustrates the distal end portion 10d with the second (primary airway) lumen 32 and with smaller first and third lumens 30, 34.

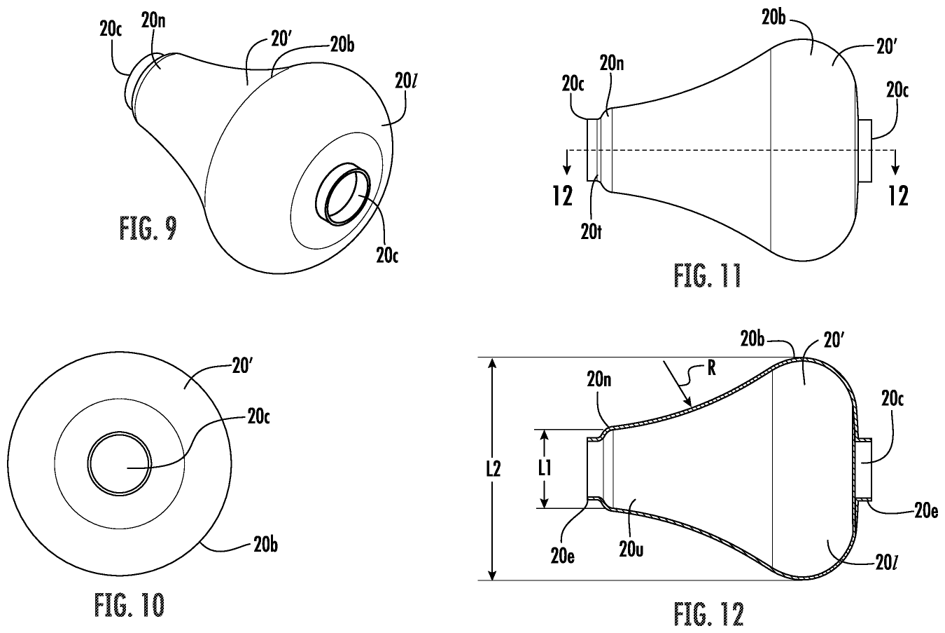

Referring to FIGS. 9-12, the cuff 20′ is shown apart from the tube 15. The cuff 20′ has a neck 20n that has a first lateral extent L1 that merges into a bottom portion segment 20b that has a second (and maximum) lateral extent L2, where L2>2(L1). L2 is typically two-three times greater than L1. The bottom portion segment 20b can have an arcuate profile or outer perimeter. The top end 20e and the bottom end 20e can each have a medial open channel 20c that is sized and configured to attach to the external wall of the tube 15. The cuff 20 can have a radius of curvature R extending between the neck 20n and the bottom portion segment 20b with the maximal lateral extent of the cuff 20′, that is in range of 1 inch to 2 inches, such as about 1.25, 1.26, 1.27, 1.28, 1.29, 1.3, 1.4, 1.5, 1.6, 1.7, 1.8, 1.9 and about 2 inches in some embodiments. A distance between the neck 20n and the bottom portion segment 20b with the maximal lateral cuff extent can be in a range of 0.5 and 1.0 inches, such as about 0.75 inches in some embodiments.

In some embodiments, the overall length of the cuff 20, 20′ can be in a range of 1-1.5 inches.

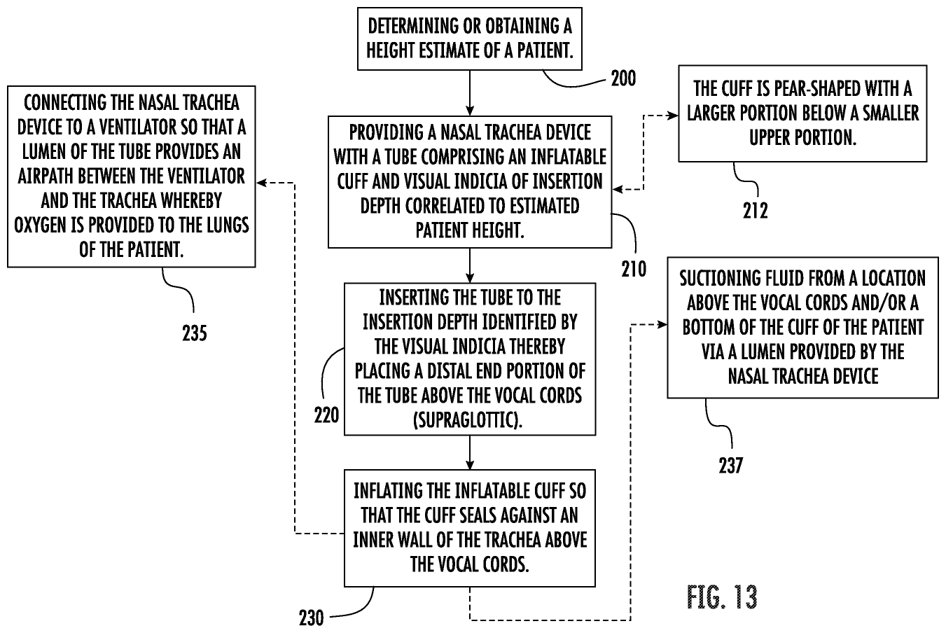

Turning now to FIG. 13, an example method of placing a nasal tracheal tube is shown. A height estimate of a patient is determined or obtained (block 200). A nasal trachea device with a tube comprising an inflatable cuff and visual indicia of insertion depth correlated to estimated patient height is provided (block 210). The tube is inserted to the insertion depth identified by the visual indicia thereby placing a distal end portion of the tube above the vocal cords (supraglottic) (block 220). The inflatable cuff is then inflated so that the cuff seals against an inner wall of the trachea above the vocal cords (block 230).

The nasal trachea device can be connected to a ventilator so that a lumen of the tube provides an airpath between the ventilator and the trachea whereby oxygen is provided to the lungs of the patient (block 235).

Fluid can be suctioned from a location above the vocal cords and/or a bottom of the cuff the patient via a lumen provided by the nasal trachea device (block 237).

The cuff can be pear-shaped with a larger portion below a smaller upper portion.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention. Therefore, it is to be understood that the foregoing is illustrative of the present invention and is not to be construed as limited to the specific embodiments disclosed, and that modifications to the disclosed embodiments, as well as other embodiments, are intended to be included within the scope of the invention.

That which is claimed:

1. A nasal tracheal device comprising:
a tube comprising a distal end portion configured to reside inside a trachea of a patient, wherein the tube has axially spaced apart proximal and distal end portions, and wherein the tube comprises a segment that resides between the proximal and distal end portions that comprises visual indicia of insertion depth correlated to patient height; and
an inflatable cuff integral to or coupled to the distal end portion of the tube, wherein the inflatable cuff is sized and configured to be positioned supraglottic.

2. The nasal tracheal device of claim 1, wherein the tube comprises a first lumen extending axially along the tube and a second lumen in fluid isolation with the first lumen and also extending axially along the tube, wherein the first lumen is in fluid communication with the inflatable cuff.

3. The nasal tracheal device of claim 1, wherein the visual indicia of insertion depth comprises a plurality of bands of different colors.

4. The nasal tracheal device of claim 1, wherein the plurality of bands comprises at least three bands of different colors, optionally 3-10 bands of different colors.

5. The nasal tracheal device of claim 1, wherein the inflatable cuff has a pear-shaped configuration or comprises a pear-shaped segment when in an inflated state.

6. The nasal tracheal device of claim 1, wherein the inflatable cuff is configured to define a fluid collection portion.

7. The nasal tracheal device of claim 2, wherein the tube comprises a third lumen extending axially along the tube in fluid isolation from the first and second lumens.

8. The nasal tracheal device of claim 2, wherein the proximal end portion of the device comprises at least one connector providing a first connection to the first lumen and a second connection to the second lumen.

9. The nasal tracheal device of claim 8, wherein the second lumen is configured to connect to a ventilator, the first lumen is configured to connect to an inflation fluid source and the third lumen is configured to connect to a vacuum source whereby the first lumen defines an inflation path, the second lumen defines an oxygen path and the third lumen defines an aspiration path.

10. The nasal tracheal device of claim 1, wherein the tube has a bend segment between the proximal and distal end portions, wherein the bend segment resides above the visual indicia of insertion depth and has an angle of between 90-120 degrees.

11. The nasal tracheal device of claim 7, wherein the third lumen is provided at least in part by a conduit that extends external to the tube with a free end that resides adjacent the cuff.

12. The nasal tracheal device of claim 11, wherein the conduit is configured to reside against a wall of the inflatable cuff when the cuff is in a non-inflated state, and wherein the conduit is configured to be pushed outward in response to inflation of the cuff.

13. The nasal tracheal device of claim 1, wherein the cuff comprises a neck at an upper end thereof that merge into a bottom portion that defines a maximal lateral extent of the inflatable cuff when inflated, and wherein the maximal lateral extent is 2-3 times greater than a lateral extent of the neck when the inflatable cuff is inflated.

14. The nasal tracheal device of claim 13, wherein the inflatable cuff has a radius of curvature that extends between the neck and the bottom portion that is in a range of 1-1.5 inches over 0.5-1 inch.

15. An intubation system comprising:
a nasal tracheal device comprising a tube with an inflatable cuff, the tube comprising a distal end portion configured to reside inside a trachea of a patient above vocal cords of the patient, wherein the tube has axially spaced apart proximal and distal end portions, wherein the tube comprises a segment that resides between the proximal and distal end portions that comprises visual indicia of insertion depth correlated to patient height, and wherein the tube comprises a first lumen extending axially along the tube and a second lumen in fluid isolation with the first lumen and also extending axially along the tube, wherein the first lumen is in fluid communication with the inflatable cuff;
a ventilator or anesthesia circuit in fluid communication with the second lumen of the tube; and
an inflation fluid source in fluid communication with the first lumen of the tube to inflate the inflatable cuff.

16. The system of claim 15, further comprising a third lumen provided by the tube and/or a conduit coupled to the tube and a vacuum in fluid communication with the third lumen and defining an aspiration path.

17. The system of claim 15, wherein the inflatable cuff comprises a pear-shaped body when in an inflated state.

18. The system of claim 15, wherein the visual indicia of insertion depth comprises a plurality of bands of different colors.

19. A method of placing a nasal tracheal tube in a patient, comprising:
determining or obtaining a height estimate of a patient;
providing a nasal trachea device with a tube comprising an inflatable cuff and visual indicia of insertion depth correlated to different heights of patients;
inserting the tube via an intranasal insertion path to an insertion depth in a trachea of the patient corresponding to the visual indicia associated with the estimated height of the patient thereby placing a distal end portion of the tube above the vocal cords (supraglottic) of the patient; and
inflating the inflatable cuff so that the cuff seals against an inner wall of the trachea above the vocal cords.

20. The method of claim 19, wherein the inserting is carried out to place a color band associated with the estimated patient height at least partially external to a nostril of the patient.

21. The method of claim 19, wherein the inflatable cuff can comprise a pear-shaped body or pear-shaped body segment with a larger portion defining a maximal lateral extent of the inflatable cuff when inflated below a smaller upper portion, wherein the larger portion is 2-3 times larger than the smaller upper portion when inflated.

22. The method of claim 19, further comprising suctioning liquid from the patient using a conduit provided by or coupled to the tube adjacent the inflatable cuff.
